(19) United States
(12) Patent Application Publication
(10) Pub. No.: US 2018/0259654 A1
(43) Pub. Date: Sep. 13, 2018

(54) INTEGRATING RADIATION DOSIMETER

(71) Applicant: Massachusetts Institute of Technology, Cambridge, MA (US)

(72) Inventors: Michael Philip Short, Cambridge, MA (US); Cody Andrew Dennett, Somerville, MA (US); Sara Elizabeth Ferry, Cambridge, MA (US); Rajiv Gupta, Wayland, MA (US)

(21) Appl. No.: 15/760,483

(22) PCT Filed: Sep. 27, 2016
(86) PCT No.: PCT/US2016/053917

§ 371 Date: Mar. 15, 2018

Related U.S. Application Data
(60) Provisional application No. 62/233,377, filed on Sep. 27, 2015.

(51) Int. Cl. G01T 1/06; G01T 1/10; A61N 5/10

(52) U.S. Cl. CPC G01T 1/06; G01T 1/10; A61N 5/1071; A61N 2005/109; A61N 2005/1087; A61N 2005/1089; A61N 2005/1074

(57) ABSTRACT

An implantable dosimeter uses salt crystals such as NaCl or KCl, or other materials that vary in color as a function of incident, ionizing radiation. The color change of the salts may occur through the creation of F-centers, where electrons become trapped in crystal defects (e.g., halide vacancies) and absorb light at certain wavelengths. Vacancies in the salt crystals absorb photons at precise wavelengths. Thus, the change in color can be correlated to the integrated dose in an implantation site. The salt crystals may be optically coupled to optical fibers or the like for remote measurement of color using, e.g., a spectrometer and a computer system. In this manner, the dosage of ionizing radiation can be measured in vivo with a fault tolerant, passively integrating dosimeter.

### CROSS-REFERENCE TO RELATED APPLICATIONS

This application is a national stage entry application of International Patent Application No. PCT/US16/53917, filed on Sep. 27, 2016, which claims priority to U.S. Provisional Patent Application No. 62/233,377, filed Sep. 27, 2015, where the entire contents of each are incorporated herein by reference.

### TECHNICAL FIELD

The present disclosure generally relates to integrating radiation dosimeters, and more specifically to fault-tolerant, passively integrating in vivo dosimeters for feedback-enabled radiation monitoring and therapy.

### BACKGROUND

For a given radiation therapy treatment, a dose of radiation supplied to the tumor is prescribed ahead of time. The patient's body is examined and scanned to allow medical staff to plan the necessary treatment process. The intensity and geometry of the beams used—in addition to the patient's placement, the use of any radiation absorbing masks, and the precise location on the skin at which the beam will be aimed—are selected with the goal of directing the prescribed dose of radiation therapy to the tumor, while minimizing radiation to surrounding healthy tissue. However, accurately determining the precise dose of radiation therapy actually delivered to the tumor during treatment remains a challenge in radiation oncology. Further, many diagnostic examinations expose patients to ionizing radiation. For example, there are image-guided interventional procedures that, in addition to exposing patients to radiation, expose operators (technologists and radiologists) to ionizing radiation while conducting these procedures. There is a need for dosimeters that enable real-time, fault-tolerant, feedback controlled radiation therapy.

### SUMMARY

An implantable dosimeter uses salt crystals such as NaCl or KCl, or other materials that vary in color as a function of incident, ionizing radiation. The color change of the salts may occur through the creation of F-centers, where electrons become trapped in crystal defects (e.g., halide vacancies) and absorb light at certain wavelengths. Vacancies in the salt crystals absorb photons at precise wavelengths. Thus, the change in color can be correlated to dose in an implantation site. The salt crystals may be optically coupled to optical fibers or other instrumentation for remote measurement of color using, e.g., a spectrometer and a computer system. In this manner, the dosage of ionizing radiation can be measured in vivo with a fault tolerant, passively integrating dosimeter.

According to one aspect, a system includes a unit of a material that exhibits a color change that varies as an integral of an amount of ionizing radiation incident on the unit, a casing about the unit of the material, and an optical coupling providing a terminal to optically couple an external device to the unit of the material.

In certain implementations, with the casing positioned in a patient, the optical coupling is accessible external to the patient for optically coupling to the external device.

In some implementations, the system further includes a fastener securable to a person to hold the casing in a relatively fixed position in proximity to the person. The fastener may be, for example, securable to skin of the person. Additionally, or alternatively, the fastener may include an adhesive, a clip or both. Further, or instead, the fastener may be securable to a garment wearable by the person.

In certain implementations, the system further includes an illumination source optically coupled to the unit of the material through the optical coupling, and an optical sensor optically coupled to the unit of the material through the optical coupling. At least one of the illumination source and the optical sensor may be releasably coupled to the optical coupling. Additionally, or alternatively, at least one of the illumination source and the optical sensor may be in a fixed position relative to the unit of the material. In certain instances, the system further includes a power source in electrical communication with the illumination source and the optical sensor.

In some implementations, the system further includes a processor coupled to and programmed to control the illumination source to illuminate the unit of material through the optical coupling, to detect one or more parameters indicative of a color of the unit of the material with the optical sensor when the unit of the material is illuminated, and to determine an exposure of the unit of the material to ionizing radiation based upon the color detected by the optical sensor. For example, the system may further include a radiation source (e.g., at least one of an ion beam, a photon beam, a neutron beam, a proton beam, an electron beam, and a heavy ion beam) configured to apply a therapeutic dose of ionizing radiation to a patient, to provide ionizing radiation with sufficient energy to induce displacements suitable for medical therapy, or both. The processor may be programmed to control operation of the radiation source based on determined exposure of the unit of the material. As an additional or alternative example, the processor may be programmed to control operation of the radiation source in a cyclic manner based on determined exposure of the unit of the material. In certain instances, the processor is programmed to control operation of the radiation source in response to detection of accumulation of radiation of the unit of the material. In some instances, the processor is programmed to determine a breathing rate of a patient based on the determined exposure of the unit of the material, and the processor is programmed to control timing of operation of the radiation source in response to the determined breathing rate.

In certain implementations, the system further includes a wireless interface for transceiving data, wherein the system is enclosed in a biocompatible enclosure sealed for wireless deployment in a patient. For example, the system may further include a wireless power receiver within the biocompatible enclosure, wherein the wireless power receiver is configured to receive power from a source external to the biocompatible enclosure and to provide power to the system. The wireless power receiver may be configured to receive power from a radiofrequency (RF) power source external to the biocompatible enclosure. Additionally, or alternatively, the system may include a wireless transceiver within the biocompatible enclosure, wherein the wireless transceiver is configured to receive power from a source external to the biocompatible enclosure to provide power to the system and to transmit an indication of an amount of ionizing radiation incident on the unit of the material. In certain instances, the illumination source is a single wavelength light source and the optical sensor includes a photodiode responsive to the single wavelength light source.

In some implementations, the illumination source includes a white light source.

In certain implementations, the material includes a salt containing one or more cations from alkali or earth metals in Groups I & II of the periodic table of elements and one or more anions from Groups III-VII of the periodic table of elements.

In some implementations, the material includes a salt consisting of one or more cations from alkali or earth metals in Groups I & II of the periodic table of elements and one or more anions from Groups III-VII of the periodic table of elements.

In certain implementations, the material includes an alkalai-halide salt.

In some implementations, the material includes potassium chloride.

In certain implementations, after a period following the color change, the material returns to an original color.

In some implementations, the unit of the material is substantially cylindrical.

In certain implementations, the unit of the material includes one or more flat faces.

In some implementations, the casing has an exterior surface suitable for in vivo implantation and use.

In certain implementations, the system further includes a plurality of units of material, each unit of the material of the plurality of units of the material exhibiting a color change that varies as an integral of an amount of incident ionizing radiation, and each unit of the material of the plurality of units of the material including a separate optical coupling for individual measurement of color change. For example, the plurality of units of the material may be arranged in a two-dimensional array transverse to an imaging axis for two-dimensional imaging of incident radiation. Additionally, or alternatively, the plurality of units may include two or more different materials each having a different color change in response to at least one of a total dose of incident radiation or a rate of incident radiation. Still further or in the alternative, the plurality of units of the material may include at least two units of the material arranged vertically along an imaging axis to provide a series of measurements at a corresponding location.

In some implementations, the casing is a biocompatible casing.

According to another aspect, a system includes a unit of a material that exhibits a color change that varies as an integral of an amount of ionizing radiation incident on the unit, an optical coupling optically connected to the unit of the material, an illumination source optically coupled to the unit through the optical coupling, and an optical sensor optically coupled to the unit through the optical coupling.

In some implementations, the system further includes a processor coupled to and programmed to control the illumination source to illuminate the unit of material through the optical coupling, to detect one or more parameters indicative of a color of the unit of the material with the optical sensor when the unit of the material is illuminated, and to determine an exposure of the unit of the material to ionizing radiation based upon the color detected by the optical sensor. In certain instances, an electrical connector may be in electrical communication with the illumination source, the optical sensor, and the processor, wherein the electrical connector is connectable in electrical communication with a power source. The electrical connector may be, for example, connectable in electrical communication with a battery.

In certain implementations, the unit of the material is positionable within a patient and the unit of material is degradable within the patient.

According to still another aspect, a system includes a unit of a material that exhibits a color change that varies as an integral of an amount of ionizing radiation incident on the unit, a casing about the unit; and an illumination source within the casing, the illumination source positioned to illuminate the unit of the material, an optical sensor within the casing positioned to detect one or more parameters indicative of a color of the unit of the material while the unit of the material is illuminated by the illumination source, and a wireless communications interface configured to transmit information from the optical sensor to a remote receiver.

In certain implementations, information includes unprocessed data from the optical sensor.

In some implementations, a system further includes a processor and a memory storing code to determine an amount of radiation exposure based upon the information from the optical sensor and to communicate the amount of radiation exposure through the wireless communications interface.

According to yet another aspect, a system includes a unit of a material that exhibits a color change that varies as an integral of an amount of ionizing radiation incident on the unit, a casing disposed about the unit of the material, and a connector coupled to the casing and releasably securable to a portable electronic device (e.g., one or more of a cellular phone, a handheld computer, an embedded system, and a mobile device) to optically couple the unit of the material to an illumination source powered by the portable electronic device and to an optical sensor powered by the portable electronic device.

In certain implementations, the connector includes an electrical coupling, the electrical coupling establishing electrical communication between a battery of the portable electronic device and one or more of the illumination source and the optical sensor when the connector is releasably secured to the portable electronic device. For example, the electrical coupling may further establish electrical communication between the battery of the portable electronic device and a processor when the connector is releasably secured to the portable electronic device, the processor coupled to one or more of the illumination source and the optical sensor. Additionally, or alternatively, the connector may define a recess releasably securable to the portable electronic device through a press fit. Still further, or instead, the connector may be releasably securable to the portable electronic device to optically couple a light emitted by the portable electronic device to the unit of the material. In certain instances, the connector may be releasably securable to the portable electronic device to optically couple a camera of the portable electronic device to the unit of the material.

According to another aspect, a system includes a unit of a material that exhibits a color change that varies as an integral of an amount of ionizing radiation incident on the unit, a casing disposed about the unit of the material, an illumination source and an optical detector within the casing, circuitry within the casing configured to control the illumination source and capture an optical signal from the optical sensor, and a connector coupled to the casing and releasably securable to an external device, the connector configured to provide power to the circuitry within the casing and to receive an electrical signal based on the optical signal from the optical sensor.

According to yet another aspect, a system includes a wearable garment, a unit of a material securable in a fixed position relative to the wearable garment, the unit of the material exhibits a color change that varies as an integral of an amount of ionizing radiation incident on the unit, and an optical coupling providing a terminal to optically couple an external device to the unit of the material.

In certain implementations, the unit of material is releasably securable to the wearable garment. For example, the unit of the material may be releasably securable to the wearable garment through the optical coupling.

In some implementations, the wearable garment includes body armor.

In certain implementations, the wearable garment includes an elastic body suit including a plurality of units of the material at predetermined locations. The predetermined locations may be selected to monitor radiation exposure during diagnostic imaging. Additionally, or alternatively, the predetermined locations may be selected to monitor radiation exposure during radiation therapy treatment.

In some implementations, the wearable garment includes a glove suitable for use by a technician or a physician during radiation therapy on a patient.

According to still another aspect, a method includes positioning a unit of a material at a location selected to measure incident radiation, wherein the material exhibits a color change that varies as an integral of an amount of ionizing radiation incident on the unit of the material, illuminating the unit of the material with a light source, detecting one or more parameters indicative of a color of the unit of the material, and determining an exposure of the unit of the material to ionizing radiation based on the color of the unit of the material.

In certain implementations, illuminating the unit of the material includes exposing the unit of the material to a broadband light source.

In some implementations, illuminating the unit of the material includes exposing the unit of the material to a single wavelength light source.

In certain implementations, the location is adjacent to tissue selected for radiation therapy. For example, the method may further include exposing the tissue to a dose of ionizing radiation from a radiation source. Additionally, or alternatively, the method may further include controlling at least one of a rate of the dose, an amount of the dose, and a direction of the dose based upon the exposure.

Other aspects, features, and advantages will be apparent from the description and drawings, and from the claims.

### BRIEF DESCRIPTION OF THE DRAWINGS

The foregoing and other objects, features and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein.

FIG. 1 depicts a system including an integrating radiation dosimeter.

FIG. 2 depicts a system including an integrating radiation dosimeter.

FIG. 3 is a schematic representation of a multi-dimensional dosimeter.

FIG. 4 is a flowchart of a method using an integrating radiation dosimeter.

FIG. 5 depicts a system including an integrating radiation dosimeter.

FIG. 6A is a front view of a system including an integrating radiation dosimeter coupled to a mobile device.

FIG. 6B is a side view of the system of FIG. 6A, taken along a cross-section of line 6B-6B in FIG. 6A.

FIG. 7 depicts a garment including an integrating radiation dosimeter.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” and the like, are words of convenience and are not to be construed as limiting terms.

Described herein are devices and systems related to, as well as techniques and methods featuring, integrating radiation dosimeters. As used herein and as described in further detail below, the term integrating radiation dosimeter includes dosimeters that detect cumulative radiation exposure passively. The integrating radiation dosimeters of the present disclosure may be interrogated (e.g., periodically interrogated) to provide an indication of cumulative radiation that has been passively detected up to the given point in time.

The integrating radiation dosimeters of the present disclosure may be useful in any of various different applications in which it is desirable to effectively and reliably detect radiation, including applications in which it may be desirable to detect integral radiation. Thus, it should be understood that the devices, systems, and methods of the present disclosure are related to integrating radiation dosimeters useful in any of various different applications in which it may be desirable to detect radiation including, by way of example and not limitation, medical treatment and diagnosis, workplace and environmental safety, and weapon detection. Accordingly, unless otherwise specified or made obvious from the context, the uses of the integrating radiation dosimeters of the present disclosure are exemplary and not limiting. For example, dosimeters of the present disclosure may include fault-tolerant, passively integrating dosimeters described as in vivo dosimeters for feedback-enabled radiation therapy, e.g., for cancer treatment and the like. Unless otherwise specified or made clear from the context, it should be understood that these fault-tolerant, passively integrating dosimeters may be used in any of various different contexts in which detecting incident radiation may be useful.

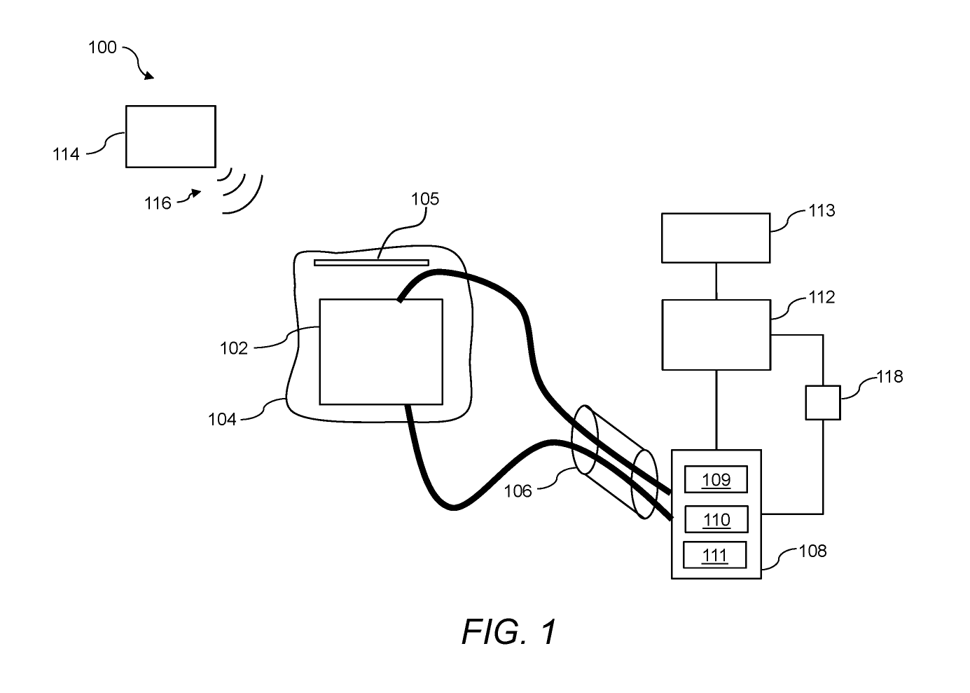

FIG. 1 depicts a system including an integrating radiation dosimeter.

The system 100 may include a unit of a material 102 (alternatively referred to herein as “the unit 102”), a casing 104, an optical coupling 106, and an external device 108.

The unit 102 may include a material that exhibits a color change that varies as an integral of an amount of ionizing radiation incident on the unit 102. The material may include a salt containing one or more cations from the alkali or earth metals in Groups I & II of the periodic table of elements and one or more elements from Groups III-VII of the periodic table of elements. The material may instead consist of one or more cations from the alkali or earth metals in Groups I & II of the periodic table of elements and one or more anions from Groups III-VII of the periodic table of elements. In one aspect, the material includes an alkalai-halide salt. The material may also or instead include sodium chloride (NaCl) or potassium chloride (KCl). By way of non-limiting examples, the elements from Groups III-VII may include elements from Groups III and IVA (e.g. (B, Al, Ga, In, Tl) and (C, Si, Ge, Sn, Pb)), or Groups III and IVB (e.g., (Sc, Y, La) and (Ti, Zr, Hf)). In another aspect the material may include any other material capable of forming F-centers when irradiated, such as certain metal oxides including, e.g., aluminum oxides or magnesium oxides. More generally, any material capable of forming an F-center, or still more generally, changing color in response to irradiation exposure, may be suitably adapted to use as a material for a sensor as contemplated herein.

Following a color change associated with exposure to irradiation, the unit 102 may return to an original color after a period of time. This return to the original color may be useful, for example, for facilitating repeated use of the unit 102. For example, during radiation therapy administered to a patient over multiple sessions (e.g., daily radiation treatments administered over multiple days or weeks), the unit 102 may return to the original color between sessions, allowing the unit 102 to be used to monitor irradiation exposure associated with each new session. The ability to monitor multiple sessions of irradiation exposure using the unit 102 can be particularly advantageous, for example, in instances in which it is desirable to implant the unit 102 to detect radiation dose incident upon a target area within a patient, and apply multiple treatments without removal and/or reinsertion of the dosimeter.

In certain implementations, the unit 102 is substantially cylindrical. The cylindrical shape of the unit 102 can, for example, facilitate packaging the unit 102 (e.g., in a size suitable for implantation in a patient using minimally invasive techniques).

In some implementations, the unit 102 has a geometry that acts as a waveguide such that color of the unit 102 may be more easily transmitted and, thus, detected. For example, the unit 102 having a geometry that acts as a waveguide may have an elongate geometry with exemplary cross-sectional geometries that include, but are not limited to, square, rectangular, circular.

The unit 102 may, additionally or alternatively, include one or more flat faces. Such flat faces can be useful, for example, for collecting incident radiation at a particular location. Also, or instead, flat faces can be useful for minimizing edge effects that can occur as the color of the unit 102 is detected.

The casing 104 may be a biocompatible casing disposed about the unit 102. The casing 104 may have an exterior surface suitable for in vivo implantation and use, such as any biocompatible rubber, thermoplastic, plastic, or the like, or any other suitable casing that can isolate the salt from surrounding physiology without obstructing incident radiation in a manner that might affect dosimetry accuracy. This may, for example, include a rubber or plastic shell encasing the salt and any related equipment, or this may include a non-permeable film or the like applied to an exterior of the salt. Alternatively, the system 100 need not include a casing, particularly where the unit 102 itself is biocompatible. For example, in such instances, the unit 102 can be implanted or otherwise delivered to the anatomy of a patient and allowed to degrade within the patient through one or more of biodegradation and bioabsorption.

In another aspect, the casing may include a fastener 105 securable to a person to hold the casing 104 in a relatively fixed position relative to the person. For example, in in vivo implementations, the fastener 105 may be securable to anatomy of the person to hold the casing 104 in a fixed or relatively fixed anatomic location (e.g., adjacent a tumor being treated using radiation therapy). Further, or instead, the fastener 105 may be securable in a relatively fixed position in proximity to the person. For example, the fastener 105 may include an adhesive such that the casing 104 may be releasably secured to the person's skin. Such placement of the casing 104 can be useful in implementations in which the radiation therapy is applied to the skin of the patient. As another, non-exclusive example, the fastener 105 may include a clip or other similar securement mechanism such that the casing 104 may be secured to a garment worn by the person. Securement of the casing 104 to a garment worn by the person can be useful, for example, in implementations in which it may be desirable to detect incident radiation on a healthcare worker in the vicinity of a patient receiving radiation therapy. In such instances, the casing 104 may be coupled to an identification badge worn by the healthcare worker and securable to an article of clothing of the healthcare worker.

The optical coupling 106 may provide a terminal to optically couple the external device 108 to the unit of material 102.

The external device 108 may include an illumination source 109, where the illumination source 109 optically couples to the unit 102 through the optical coupling 106, which may be a fiber optic, fiber bundle, or any suitable combination of transducers and hardware for transmitting illumination and receiving optical data from the casing 104. The illumination source 109 may include a white light source or the like.

The system 100 may further include an optical sensor 110 optically coupled to the unit 102 through the optical coupling 106. The optical sensor 110 may include, for example, an optical spectrometer for measuring properties of light reflected from and/or transmitted through the unit 102. That is, more generally, the optical sensor 110 may measure one or both of the transmitted and reflected spectra. Additionally, or alternatively, the optical sensor 110 may include a photodiode. More generally, the optical sensor 110 is suitable for sensing light reflected from the unit 102 as the unit 102 is illuminated by the illumination source 109. Accordingly, the type and arrangement of the optical sensor 110 may depend on various different factors including, by way of example, the type of illumination source 109, size requirements, power consumption limitations, etc.

The unit 102 and at least a portion of the casing 104 may be positioned in the patient (e.g., adjacent a treatment site), and the optical coupling can be accessible external to the patient for coupling to the external device 108. Additionally, or alternatively, the illumination source 109, the optical sensor 110, or both may be releasably coupled to the optical coupling 106. For example, one or both of the illumination source 109 and the optical sensor 110 can be releasably coupled to the optical coupling through an interference fit. Advantageously, a releasable coupling may facilitate disconnecting the illumination source 109, the optical sensor 110, or both from the unit 102 between treatments to minimize interference with the patient when the patient is not receiving radiation therapy.

The external device 108 may further, or instead, include a power source 111 in electrical communication with one or both of the illumination source 109 and the optical sensor 110. The power source 111 may be any of various different portable power sources including, without limitation, one or more batteries. Thus, for example, in instances in which the illumination source 109 and the optical sensor 110 are releasably coupled to the optical coupling 106, the illumination source 109, the optical sensor 110, and the power source 111 can be packaged in a portable unit (e.g., a handheld unit).

The system 100 may also include a processor 112 coupled to and programmed to control the external device 108. In one aspect, the processor 112 is programmed to control the illumination source 109 to illuminate the unit of material 102 through the optical coupling 106, to detect a color of the unit 102 with the optical sensor 110 when illuminated, and to determine an exposure of the unit of material 102 to ionizing radiation based upon the color.

The system 100 may include a memory 113 in communication with the processor 112. The memory 113 may store code to determine an amount of radiation exposure of the unit 102. In use, the processor 112 may receive information from the optical sensor 110 and may execute the code stored in the memory 113 to determine an amount of radiation exposure of the unit 102. Additionally, or alternatively, the processor may communicate the amount of radiation exposure through a wireless communications interface, such as any of the various different wireless interfaces described herein.

The system 100 may include an electrical connector 118 in electrical communication with the external device 108 and the processor 112. The electrical connector 118 may be connectable in electrical communication with a power source to power to the external device 108 and the processor 112, as the case may be. For example, the electrical connector 118 may be connectable in electrical communication with a battery such as an external battery or, in implementations in which the external device includes the power source 111, in communication with the power source 111 to charge the power source 111. Thus, continuing with this example, the optical coupling 106, the external device 108, the processor 112, and the memory 113 may be carried in or on a single housing to facilitate portability. That is, a housing containing the optical coupling 106, the external device 108, the processor 112, and the memory 113 can be handheld and, optionally, reusable for connection to various different units of material.

The system 100 may include a radiation source 114 configured to apply a therapeutic dose of ionizing radiation 116 to a patient. The radiation source 114 may include a source to supply at least one of an ion beam, a photon beam, a neutron beam, a proton beam, an electron beam, and a heavy ion beam. In an aspect, the radiation source 114 provides ionizing radiation 116 with sufficient energy to induce atomic displacements suitable for medical therapy. As shown in the figure, the radiation source 114 may be provided separate from the external device 108. In another aspect, the radiation source 114 may also or instead be included in the external device 108.

In certain instances, the accuracy of the radiation dose delivered from the radiation source 114 to target tissue can be impacted by the patient's breathing, or movement of other internal organs. That is, the patient's regular breathing, digestion, and any other involuntary or voluntary movements can result in movement of the target tissue with respect to the path of radiation from the radiation source 114. To reduce dosing inaccuracy that can result from such unavoidable movement, the processor 112 may be programmed to determine whether the treatment radiation is actually being applied to the target area, either in a cyclic manner (without direct feedback) or by monitoring and responding to the accumulation of radiation (with direct feedback). For example, the breathing rate of the patient may be determined based on an observed periodic variation of detected radiation incident upon the unit 102. The processor 112 may be programmed to control timing of operation of the radiation source 114 in response to the determined breathing rate. For example, the processor 112 may control timing of operation of the radiation source 114 such that radiation is delivered with a periodicity corresponding to a specific portion of the breathing cycle of the patient (e.g., the portion of the cycle corresponding to maximum exhalation).

In an aspect, the processor 112 is programmed to control operation of the radiation source 114 in response to the exposure of the unit of material 102.

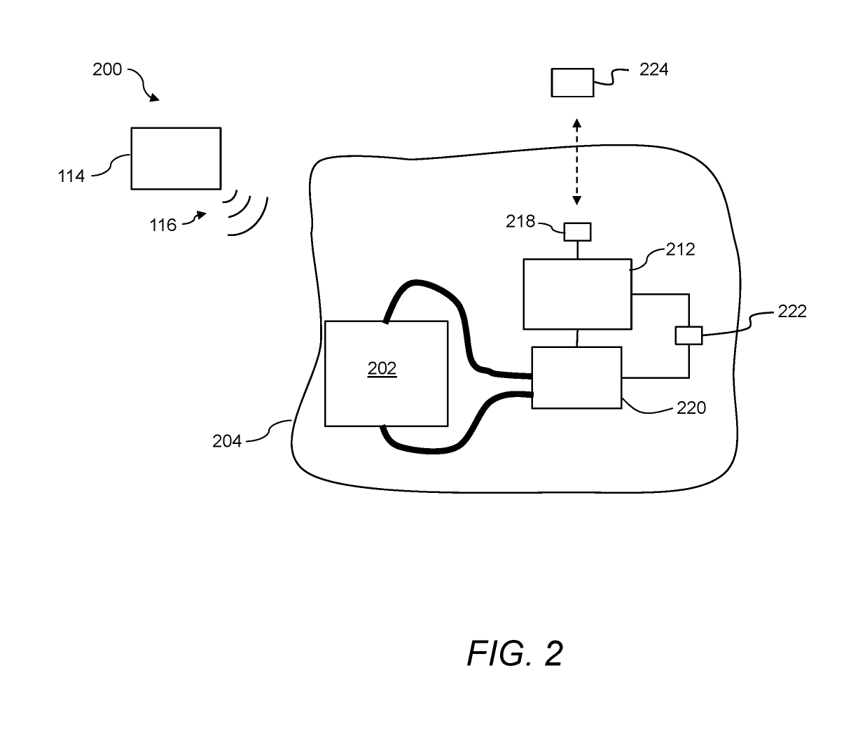

FIG. 2 depicts a system including an integrating radiation dosimeter. A system 200 may be generally as described above, except that optical components 220 (such as the illumination source and optical sensor, may be contained within an enclosure 204, along with a unit of material 202. The unit of material 202 may include a material that exhibits a color change that varies as an integral of an amount of ionizing radiation incident on the unit 202 and, thus, may include any of the various different materials and/or features of the unit of material 102 (FIG. 1).

The enclosure 204 may be formed of any of the various different types of materials described herein with respect to the casing 104 (FIG. 1). Accordingly, the enclosure 204 may be a biocompatible material such that the enclosure 204 may be implanted or otherwise delivered to a target treatment area within a patient during the course of a radiation therapy.

In one aspect, one or more of the optical components 220 such as the illumination source and/or optical sensor may be chip-based devices implanted directly within or adjacent to the material and electrically coupled to the processor for illumination and data acquisition. The biocompatible enclosure 204 may also contain a wireless communications interface 218 so that the system 200 or subcomponents thereof can be sealed within the biocompatible enclosure 204 for wireless deployment in a patient. For example, the system 200 or components thereof (e.g., the optical components 220) can be fixed relative to one another with the biocompatible enclosure 204 permanently sealed to facilitate withstanding a variety of different environments upon implantation or deployment in any of various different non-medical applications. As another example, the biocompatible enclosure 204 may be resealable such that any of various different components of the system 200 may be replaced or repaired.

The system 200 may also include a wireless power receiver 222 within the biocompatible enclosure 204 configured to wirelessly receive power from an external source (using, e.g., any suitable RF or magnetic resonance technology known in the art) and provide power to the system 200 or subcomponents thereof. The wireless communications interface 218 may generally be configured to communicate with a remote receiver 224 using any suitable short range communications interface including by way of example, WiFi or any other IEEE 802.xx protocol, Near Field Communications, BlueTooth, or any other radio frequency or similar wireless communications technique(s). For example, the wireless communications interface 218 may transmit unprocessed data from the optical components 220 such that a processor, external to the biocompatible enclosure 204, may process the information from the optical components 220 to determine an amount of incident radiation. Such external processing can reduce the size and number of components required to be enclosed in the biocompatible enclosure 204 and, thus, can facilitate reducing the size of the biocompatible enclosure 204. It should be appreciated, therefore, that external processing can facilitate sizing the biocompatible enclosure 204 for implantation and/or delivery to anatomic regions that may otherwise be inaccessible with a larger device.

Additionally, or alternatively, the optical components 220 can include a single wavelength light source and a photodiode responsive to the single wavelength light source. In use, the single wavelength light source may be powered to act as an illumination source for the unit of material 202, and the photodiode may be powered to detect color of the unit of material 202. The power and size requirements associated with such an arrangement of a single wavelength light source and a photodiode can be useful, for example, for accurate determination of incident radiation within a small footprint suitable for implantation or other forms of delivery to various different parts of the anatomy that are treatable using radiation therapy.

In another aspect, the wireless interface 218 may include a transceiver such that the wireless communications interface 218 may transceive data such as control information, status information, diagnostics, an identification number, an amount of ionizing radiation incident on the unit of the material, and so forth. The transceiver may receive power from the wireless power receiver 222 to transmit data from within the biocompatible enclosure 204 to the remote receiver 224.

More generally, any combination of optical components, electronics components, communication components, and processing components may be employed according to various configurations for wireless or wired use, autonomous or monitored/controlled use, and so forth.

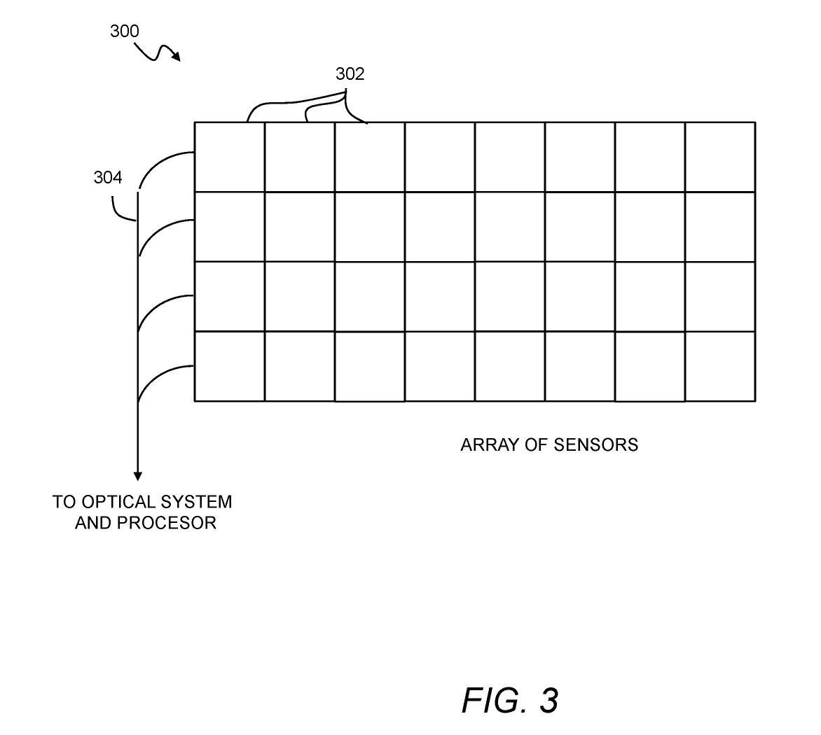

FIG. 3 is a schematic representation of a multi-dimensional dosimeter. One skilled in the art will recognize that the systems depicted in FIGS. 1 & 2 may also or instead include a plurality of units of material. For example, in one aspect, a system 300 includes a plurality of units 302 of material that exhibit a color change that varies as an integral of an amount of incident ionizing radiation incident, where each unit includes a separate optical coupling 302 for individual measurement of color change. The plurality of units may be arranged in a two-dimensional array transverse to an imaging axis for two-dimensional imaging of incident radiation. This may include any pattern and density of individual measurement cells consistent with physical spacing limitations and the desired spatial resolution. In one aspect, a 2D array of sensors (e.g., individual units of salt(s)) may be arranged on a substrate that maintains a desired arrangement of crystals during deployment and use. The plurality of units may also or instead include at least two units arranged vertically along an imaging axis to provide a series of measurements corresponding locations, e.g., varying depths into an irradiated target. This capability may facilitate improved measurement and analysis of patterns of incident radiation that may be particularly useful where, e.g., the 2D intensity pattern of a radiation source is unknown or the target is a heterogeneous mass that might respond differently to radiation at different locations and depths. In another aspect, the plurality of units 150 may include two or more different materials each having a different color change in response to at least one of a total dose of incident radiation or a rate of incident radiation. Thus the system may usefully provide a variety of 2D and/or 3D measurements of the amount and/or rate of incident radiation in an area of interest.

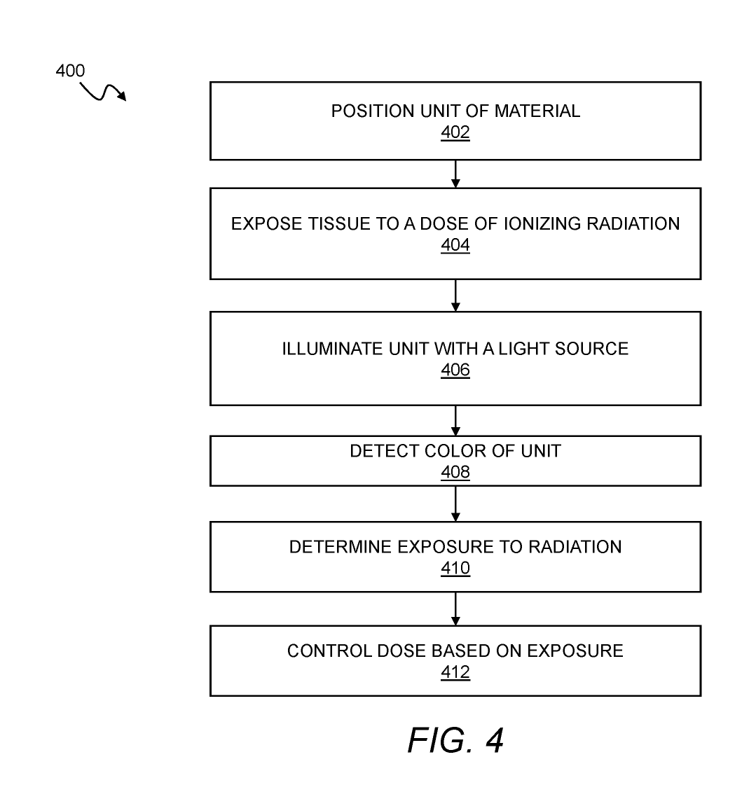

FIG. 4 is a flowchart of a method using an integrating radiation dosimeter.

As shown in step 402, the method 400 may include positioning a unit of a material at a location selected to measure incident radiation. This may, for example, be a location within tissue that will receive irradiation, or for 2D imaging, in a plane within or beneath the target body. Position will more generally depend on the region of interest and the desired spatial resolution. The material may be any of the materials described herein that exhibit a color change that varies as an integral of an amount of ionizing radiation incident on the unit. The location may, for example, be adjacent to or embedded within tissue selected for radiation therapy.

As shown in step 404, the method 400 may include exposing the tissue to a dose of ionizing radiation from a radiation source. This may, for example, include any source of radiation including any therapeutic or diagnostic radiation source useful for treating or imaging human tissue.

As shown in step 406, the method 400 may include illuminating the unit with a light source (e.g., a broadband light source, a single wavelength light source, and combinations thereof). This may, for example, be an incandescent light source, an LED light source, a laser light source, or any other light source suitable for illuminating the material through an optical coupling with a range of wavelengths covering the range of interest, e.g., the range of wavelengths where F-center vacancies will selectively absorb incident narrowband or broadband illumination.

As shown in step 408, the method 400 may include detecting one or more parameters indicative of a color of the unit. In general, the detection of one or more parameters indicative of a color in step 408 may depend upon the type of illumination source used in step 406. Further, as used herein, the detection of one or more parameters indicative of a color in step 408 shall be understood to include measuring one or more parameters of the unit in response to the illumination source and, additionally or alternatively, the one or more parameters shall be understood to include parameters directly or indirectly related or correlated to color of the unit. Thus, for example, the detection of color in step 408 may include a direct measurement of color of the unit. Further, or instead, the detection of color of the unit may be based on comparing the one or more parameters to corresponding one or more parameters for a known color, as described in further detail below.

For example, in implementations in which the unit is illuminated with a broadband light source, detecting the one or more parameters indicative of color of the unit may include measuring the amount of absorption at particular wavelengths by comparing the broadband spectrum from an unexposed crystal and an exposed crystal. Continuing with this example, notches will appear in the spectrum corresponding to the F-center wavelength, and one or more characteristics may be determined, including spectral positions of multiple notches, if appropriate, the width of each notch, and the overall attenuation in light transmission.

In addition, or in the alternative, in implementations in which the unit is illuminated with a single wavelength light source, detecting the one or more parameters indicative of color of the unit may include tuning each single wavelength to a known color center wavelength.

As shown in step 412, the method 400 may include determining an exposure of the unit to ionizing radiation based on the color of the unit. For example, in implementations in which the illumination source is a broadband light source, determining an exposure of the unit to ionizing radiation may include correlating one or more detected parameters to a particular exposure level. Examples include correlating one or more of spectral positions of multiple notches, if appropriate, the width of each notch, and the overall attenuation in light transmission to a particular exposure level. As another non-limiting example, in implementations in which the illumination source is a single wavelength light source, as described above, determining an exposure of the unit to ionizing radiation may include correlating the simple attenuation of each single wavelength.

It will be appreciated that some steps in the method 400 contemplated herein are dependent on other steps, while others are not. For example, detecting the one or more parameters indicative of a color presupposes an incident illumination such as the broadband illumination and/or the single wavelength light source contemplated in step 406. However, this illumination/measurement sub-process may in principle be performed at any time independently from the irradiation step. That is, the detection of one or more parameters indicative of a color may be during and/or after irradiation, or any combination of these. While it may be desirable to detect the color change around the time of irradiation where, for example, the color change is reversible and decays over time. And in some instances a particular time period for measurement may be of greater diagnostic significance. As another example, it may be useful to provide a measure of radiation dosage in real time during a procedure, or this may only be relevant for post-irradiation analysis, and as such the final determination step to measure exposure may be suitably deferred in some contexts. However, absent these types of circumstances the two main sub-processes (irradiation and detection) are generally independent.

As shown in step 412, the method 400 may include controlling an aspect of the dose based upon the exposure. This may include controlling at least one of a rate of the dose, an amount of the dose, and a direction of the dose based upon the exposure.

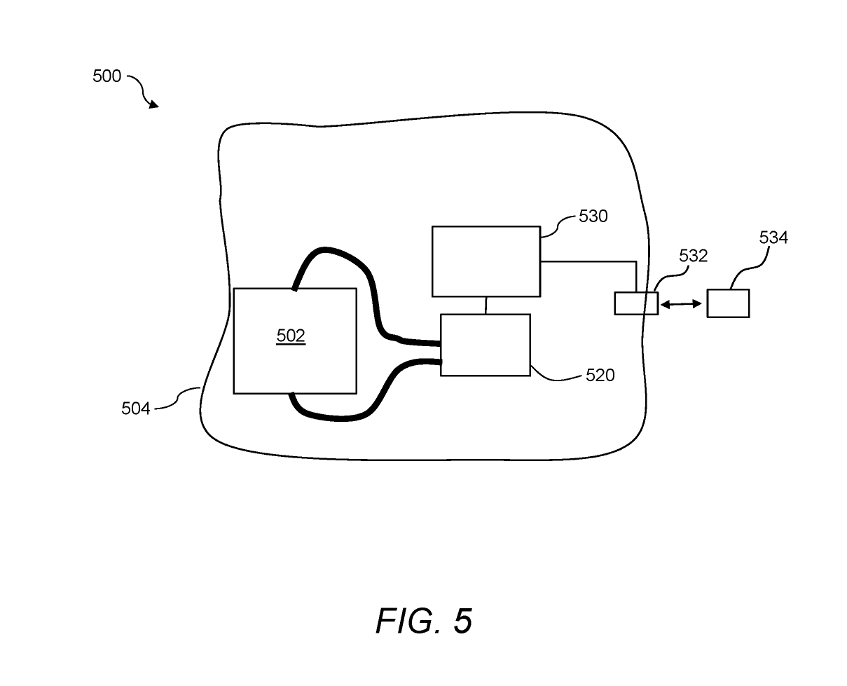

FIG. 5 depicts a system including an integrating radiation dosimeter. A system 500 may include a unit of material 502, a casing 504, optical components 520, circuitry, a connector 532, and an external device 534. Within the casing 504, the unit of material 502 may be optically coupled to the optical components 520, and circuitry 530 may be in electrical communication with the optical components and a connector 532 coupled to the casing 504. The unit of material 502 may be generally as described above with respect to material that exhibits a color change that varies as an integral of an amount of ionizing radiation incident on the unit 502. The casing 504 may be generally as described above and, thus, may include any one or more of the various different features described above with respect to the casing 104 and/or the enclosure 204. Similarly, the optical components 520 may be generally as described above with respect to the optical components 220 and, thus, may include any number and combination of illumination sources and optical sensors described herein.

The circuitry 530 may be in electrical communication with the optical components 520 such that the circuitry controls an illumination source and captures an optical signal from an optical sensor. For example, the circuitry 530 may turn the illumination source on and off, based on power provided to the circuitry. Additionally, or alternatively, the circuitry 530 may coordinate capturing an optical signal from the optical sensor in coordination with illumination provided to the unit of material 502 by the illumination source.

The connector 532 may be releasably securable to an external device 534, which may include a power source 534. The connector 532 may be in electrical communication with the circuitry 530 such that an electrical signal passing through the connector 532 may power the optical components 520. The circuitry 530 may return to the connector 532 an electrical signal based on the optical signal from the optical sensor and, with the external device 534 connected to the connector 532, the electrical signal based on the optical signal may be passed to the external device 534.

The external device 534 may be a handheld reader that can be plugged into the connector 532 to receive an electrical signal indicative of an integral of an amount of ionizing radiation incident on the unit 502. Additionally, or alternatively, the external device 534 may be a mobile computing device (e.g., a mobile phone). The connector 532 may be releasably securable to the external device 534 through a standardized connection on the mobile computing device (e.g., a headphone jack, a USB port, and/or other similar bus connectors).

In use, the external device 534 may be used to plug into the connector 532 to provide an on-demand indication of radiation exposure of the unit of material 502 associated with the connector 532. For example, in an emergency situation in which many individuals may have been exposed to potentially dangerous radiation levels, the external device 534 may be plugged into connectors 534 associated with respective individuals, and the resulting readings made by the external device 534 may be useful for triaging the individuals according to levels of radiation exposure.

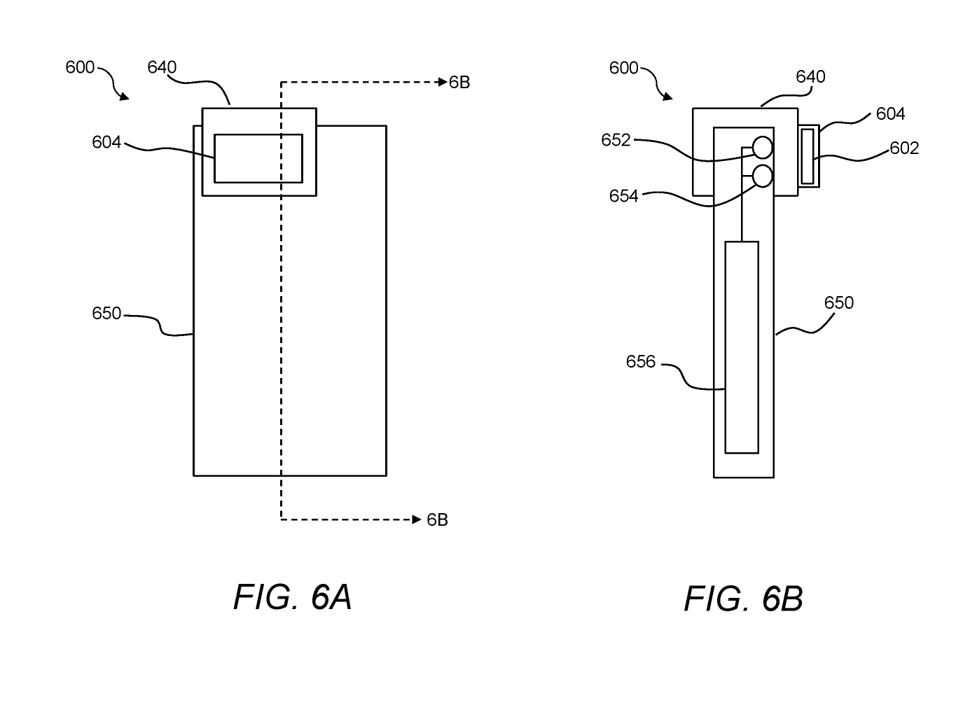

FIG. 6A is a front view of a system including an integrating radiation dosimeter coupled to a mobile device. FIG. 6B is a side view of the system of FIG. 6A, taken along a cross-section of line 6B-6B in FIG. 6A.

A system 600 may include a connector 640 coupled to a portable electronic device 650. For example, the connector 640 may define a recess positionable on the portable electronic device 650 through a press or interference fit. As an example, the connector 640 may be slid onto an edge of the portable electronic device 650 for use and slid from the edge of the portable electronic device 650 between uses. In general, the portable electronic device 650 may be a ubiquitous computing device including a power source and a processor and, thus, for example, may include one or more of a cellular phone, a handheld computer, an embedded system, and a mobile device.

The connector 640 may be coupled to a casing 604 carrying a unit of material 602. The casing 604 may be generally as described above and, thus, may include any one or more of the features of the casings 104, 204, and 504 described above with respect to FIGS. 1, 2, and 5. Similarly, the unit of material 602 may be generally as described above and, thus, may include any one or more of the features of the units 102, 202, and 502 described above with respect to FIGS. 1, 2, and 5.

The mobile device 650 may be a cellular phone or other similar device including an illumination source 652 and an optical sensor 654, such as may be part of a camera of the mobile device 650. That is, the illumination source 652 may include a flash of the camera of the mobile device 650 and, additionally or alternatively, the optical sensor 654 may include a lens of the camera of the mobile device. In such implementations, the connector 640 may be a passive device coupled to the casing 604, and the connector 640 may be releasably securable to the mobile device 650 such that the camera of the mobile device 650 is optically coupled to the unit 602 carried by the connector 640. Thus, in use, the illumination source 652 of the camera of the mobile device 650 may direct light toward the unit of material 602 and an optical sensor 654 of the camera of the mobile device 650 may capture information indicative of the color of the unit 602 upon illumination. In certain implementations, the information captured by the optical sensor 654 of the mobile device 650 may be analyzed by an application resident on the mobile device 650. Additionally, or alternatively, the information captured by the optical sensor 654 of the mobile device 650 may be transmitted (e.g., via a wireless communication network) to a remote processor for analysis.

In general, the mobile device 650 may carry a battery 506. Thus, while the mobile device 650 itself may carry the illumination source 652 and/or the optical sensor 654, it should be appreciated that one or both of the illumination source 652 and the optical sensor 654 may be disposed on the connector 640, and electrical power may be provided to the connector 640 via an electrical coupling between the connector 640 and the battery 506. The electrical coupling may include, for example, an electrical connection between the connector 640 and of the various different connectors available on the mobile device 650, including, without limitation, a headphone jack, a USB port, or other similar a bus connection. In addition, or as an alternative to establishing electrical communication between the connector 640 to the battery 506, the electrical coupling may establish electrical communication between the connector 640 and a processor carried by the mobile device 650 such that, for example, the processor may control the illumination source 652 and process a corresponding signal received from the optical sensor 654 to provide a measurement or other indication of incident radiation on the unit 602.

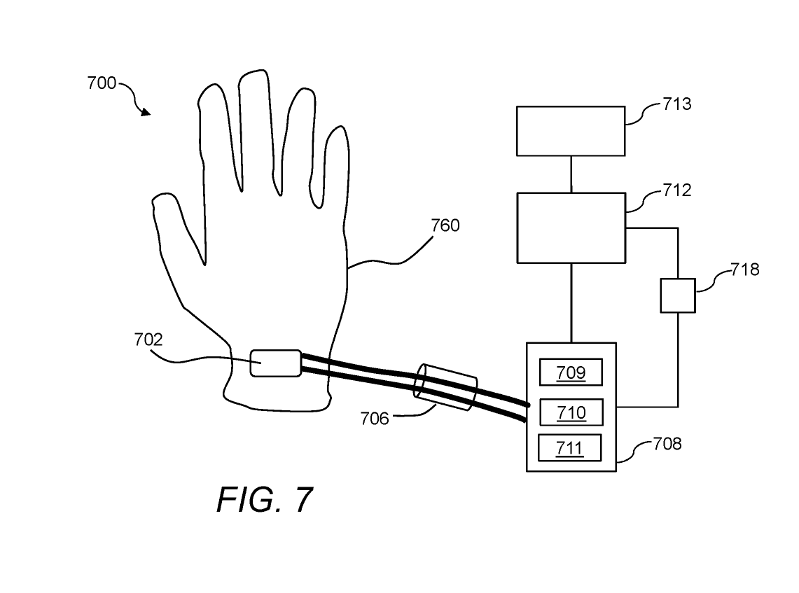

FIG. 7 depicts a garment including an integrating radiation dosimeter.

A system 700 may include a unit of material 702, an optical coupling 706, and a wearable garment 760. The unit of material 702 may be generally as described above and may, optionally, be enclosed in a casing such as any of the casings or enclosure described above. Similarly, the optical coupling 706 may be generally as described above and, thus, may provide a terminal to optically couple an external device 708 to the unit of material 702 according to any one or more of the various different methods described herein. More generally, element numbers of the system 700 may be similar to corresponding element numbers in the system 100 in FIG. 1. Thus, as an example, it should be understood that the external device 708 may be generally as described above with respect to the external device 108, unless otherwise specified or made clear from the context.

The unit of material 702 may be securable in a fixed position relative to the wearable garment 760 such that the unit of material exhibits a color change that varies as an integral of an amount of ionizing radiation incident on the unit and, thus, provides an indication of ionizing radiation incident on the wearable garment 760. For example, the unit of material 702 may be releasably securable to the wearable garment 760. As an example, the unit of material 702 may be releasably securable to the wearable garment 760 via a connector such that the unit of material 702 may be remove from the wearable garment 760 for purpose of replacing the unit of material 702, care/washing of the wearable garment 760, etc. Additionally, or alternatively, the unit of material 702 may be releasably securable to the wearable garment 760 through the optical coupling 706. That is, in addition to optically coupling the unit of material 702 to an illumination source and an optical sensor, the optical coupling 706 may provide mechanical engagement between the optical coupling 706 and the wearable garment 760.

The wearable garment 760 may be any of various different types of garments worn by a person during circumstances related to radiation exposure. In general, the type of garment and the position of the unit of material 702 on the wearable garment 760 may be dependent upon the context in which it is desirable to monitor incident radiation on the garment.

In a medical provider context, the wearable garment 760 may be a glove (e.g., a latex or nitrile surgical glove) worn by a healthcare provider (e.g., a physician, nurse, technician, etc.) during radiation therapy on a patient. Such a glove may be useful, for example, for alerting the healthcare provider of inadvertent exposure of the healthcare provider to radiation intended for the patient.

In a medical treatment and/or diagnosis context, the wearable garment 760 may include a body suit (e.g., an elastic body suit easily positionable on a patient) including a plurality of units of material 702 disposed at predetermined locations. The predetermined locations may be selected, for example, to monitor radiation exposure during radiation-based diagnostic imaging (e.g., x-ray, computed tomography (CT) scans, molecular imaging, and the like). Additionally, or alternatively, the predetermined locations may be selected to monitor exposure of the patient to radiation during radiation therapy. Thus, in general, the wearable garment 760 may include a body suit including a plurality of units of material 702 disposed at predetermined locations to monitor exposure at those predetermined locations during medical treatment, diagnosis, or both, such that appropriate precautions can be taken to mitigate the risk of exposing healthy tissue of the patient to dangerous levels of radiation.

In a military context, the wearable garment 760 may include body armor suitable for protecting a soldier from bullets or other projectiles associated with warfare. It should be understood that, in this context, securing the unit of material 702 to the wearable garment 760 may provide the soldier with additional protection, in the form of early detection, from radiation-based weapons.

While integrating, fault tolerant radiation dosimeters have been described as being in casings, enclosures, and/or in garments, it should be appreciated that additional or alternative implementations are possible. As an example, one or more of the salts described herein can be incorporated into paint or otherwise distributed on a surface (e.g., walls) of a medical facility, medical equipment, or any other facility that uses or processes radioactive materials (such as a nuclear reactor) to monitor incident radiation on the surface. Continuing with this example, an illumination source and an optical sensor (e.g., combined into an external device according to any one or more of the various implementations described herein) can be used in combination to detect color change of the one or more salts on the surface of the medical or other facility to provide an indication of incident radiation on the surface. This can be useful, for example, for improving safety of medical personnel in the vicinity of radiation therapy, or radiation workers in all areas of a nuclear power plant or radiation laboratory.

In general, the foregoing description contemplates an integrating, fault tolerant radiation dosimeter with significant advantages over other systems and methods known in the art.

The disclosed systems and methods are deployable in vivo. That is, the sensor can be implanted at any desired location within a radiation target to permit measurement at the precise location of interest rather than inferring exposure based on an external measurement. Further, the use of common salts render the material generally safe and biocompatible, which advantageously mitigates risks of exposure to toxic or otherwise harmful materials even in the event that a casing or other container becomes damaged or ruptures during use.

The disclosed systems and methods provide real time measurement capabilities. That is, the system does not depend on post-processing or other complex computation to arrive at accurate measurements. Instead, exposure can be determined immediately based on the measured color of a salt-based sensor.

The disclosed systems and methods provide an integrated measurement and/or an integrating sensor. In particular, a color change of the type contemplated here—color change induced by F-center vacancies—is a cumulative change that accompanies an accumulation of F-center vacancies resulting from irradiation. Thus it is possible to evaluate the accumulated or integrated amount of radiation exposure based on a single color measurement. While the device is inherently integrating in nature, based on the underlying physical phenomena that drive the measurement, there are also a variety of techniques for capturing instantaneous exposure or exposure rate information. For example, a time sequence of integrated measurements may be taken and used to infer a rate of exposure for each time interval. In another aspect, a number of different salts or the like that have different exposure rate responses may be used concurrently to infer an instantaneous rate of exposure.

The disclosed systems and methods support fault tolerant measurement. For example, the integrating nature of the underlying physical process is independent of monitoring or measurements. Thus, even if measurement hardware coupled to a sensor fails during a procedure, the sensor itself will nonetheless capture an integrated measurement of exposure in a number of optically active vacancies, F-centers or other crystalline or amorphous defects. The sensor can be tested for color change after a procedure using new or repaired optical hardware without a significant loss of information. Thus, each sensor provides a fault-tolerant measurement of exposure independent of the hardware used to quantitatively measure the exposure.

Numerous other advantages will readily be appreciated. The hardware is inexpensive and generally safe for medical/biological use. The quantitative measurements of exposure require relatively simple calculations that can be performed in real time (e.g., without observable latency) using commonly available optical hardware and processing resources. And the physical sensors scale easily into 2D or 3D measurement arrays.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof, and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity, and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A system comprising:
a unit of a material that exhibits a color change that varies as an integral of an amount of ionizing radiation incident on the unit;
a casing about the unit of the material; and
an optical coupling providing a terminal to optically couple an external device to the unit of the material.

2. The system of claim 1 wherein, with the casing positioned in a patient, the optical coupling is accessible external to the patient for optically coupling to the external device.

3. The system of any one of claims 1-2 further comprising a fastener securable to a person to hold the casing in a relatively fixed position in proximity to the person.

4. The system of claim 3 wherein the fastener is securable to skin of the person.

5. The system of any one of claims 3-4 wherein the fastener includes an adhesive.

6. The system of any one of claims 3-5 wherein the fastener is securable to a garment wearable by the person.

7. The system of claim 6 wherein the fastener includes a clip.

8. The system of any one of claims 1-7 further comprising:
an illumination source optically coupled to the unit of the material through the optical coupling; and
an optical sensor optically coupled to the unit of the material through the optical coupling.

9. The system of claim 8 wherein at least one of the illumination source and the optical sensor is releasably coupled to the optical coupling.

10. The system of claim 8 wherein at least one of the illumination source and the optical sensor is in a fixed position relative to the unit of the material.

11. The system of any one of claims 8-10 further comprising a power source in electrical communication with the illumination source and the optical sensor.

12. The system of any one of claims 8-11 further comprising a processor coupled to and programmed to control the illumination source to illuminate the unit of material through the optical coupling, to detect one or more parameters indicative of a color of the unit of the material with the optical sensor when the unit of the material is illuminated, and to determine an exposure of the unit of the material to ionizing radiation based upon the color detected by the optical sensor.

13. The system of claim 12 further comprising a radiation source configured to apply a therapeutic dose of ionizing radiation to a patient.

14. The system of claim 13 wherein the processor is programmed to control operation of the radiation source based on determined exposure of the unit of the material.

15. The system of claim 14, wherein the processor is programmed to control operation of the radiation source in a cyclic manner based on determined exposure of the unit of the material.

16. The system of any one of claims 14 and 15, wherein the processor is programmed to control operation of the radiation source in response to detection of accumulation of radiation of the unit of the material.

17. The system of any one of claims 14-16 wherein the processor is programmed to determine a breathing rate of a patient based on the determined exposure of the unit of the material, and the processor is programmed to control timing of operation of the radiation source in response to the determined breathing rate.

18. The system of any one of claims 13-17 wherein the radiation source is at least one of an ion beam, a photon beam, a neutron beam, a proton beam, an electron beam, and a heavy ion beam.

19. The system of any one of claims 13-18 wherein the radiation source provides ionizing radiation with sufficient energy to induce atomic displacements suitable for medical therapy.

20. The system of any one of claims 8-19 further comprising a wireless interface for transceiving data, wherein the system is enclosed in a biocompatible enclosure sealed for wireless deployment in a patient.

21. The system of claim 20 further comprising a wireless power receiver within the biocompatible enclosure, wherein the wireless power receiver is configured to receive power from a source external to the biocompatible enclosure and to provide power to the system.

22. The system of claim 21 wherein the wireless power receiver is configured to receive power from a radiofrequency (RF) power source external to the biocompatible enclosure.

23. The system of any one of claims 20-22 further comprising a wireless transceiver within the biocompatible enclosure, wherein the wireless transceiver is configured to receive power from a source external to the biocompatible enclosure to provide power to the system and to transmit an indication of an amount of ionizing radiation incident on the unit of the material.

24. The system of claim 23 wherein the illumination source is a single wavelength light source and the optical sensor includes a photodiode responsive to the single wavelength light source.

25. The system of any one of claims 8-24 wherein the illumination source includes a white light source.

26. The system of any one of claims 1-25 wherein the material includes a salt containing one or more cations from alkali or earth metals in Groups I & II of the periodic table of elements and one or more anions from Groups III-VII of the periodic table of elements.

27. The system of any one of claims 1-26 wherein the material includes a salt consisting of one or more cations from alkali or earth metals in Groups I & II of the periodic table of elements and one or more anions from Groups III-VII of the periodic table of elements.

28. The system of any one of claims 1-27 wherein the material includes an alkalai-halide salt.

29. The system of any one of claims 1-28 wherein the material includes potassium chloride.

30. The system of any one of claims 1-29 wherein, after a period following the color change, the material returns to an original color.

31. The system of any one of claims 1-30 wherein the unit of the material is substantially cylindrical.

32. The system of any one of claims 1-31 wherein the unit of the material includes one or more flat faces.

33. The system of any one of claims 1-32 wherein the casing has an exterior surface suitable for in vivo implantation and use.

34. The system of any one of claims 1-33 further comprising a plurality of units of material, each unit of the material of the plurality of units of the material exhibiting a color change that varies as an integral of an amount of incident ionizing radiation, and each unit of the material of the plurality of units of the material including a separate optical coupling for individual measurement of color change.

35. The system of claim 34 wherein the plurality of units of the material is arranged in a two-dimensional array transverse to an imaging axis for two-dimensional imaging of incident radiation.

36. The system of any one of claims 34-35 wherein the plurality of units include two or more different materials each having a different color change in response to at least one of a total dose of incident radiation or a rate of incident radiation.

37. The system of any one of claims 34-36 wherein the plurality of units of the material includes at least two units of the material arranged vertically along an imaging axis to provide a series of measurements at a corresponding location.

38. The system of any one of claims 1-37 wherein the casing is a biocompatible casing.

39. A system comprising:
a unit of a material that exhibits a color change that varies as an integral of an amount of ionizing radiation incident on the unit;
an optical coupling optically connected to the unit of the material;
an illumination source optically coupled to the unit through the optical coupling; and
an optical sensor optically coupled to the unit through the optical coupling.

40. The system of claim 39 further comprising a processor coupled to and programmed to control the illumination source to illuminate the unit of material through the optical coupling, to detect one or more parameters indicative of a color of the unit of the material with the optical sensor when the unit of the material is illuminated, and to determine an exposure of the unit of the material to ionizing radiation based upon the color detected by the optical sensor.

41. The system of claim 40 further comprising an electrical connector in electrical communication with the illumination source, the optical sensor, and the processor, wherein the electrical connector is connectable in electrical communication with a power source.

42. The system of claim 41 wherein the electrical connector is connectable in electrical communication with a battery.

43. The system of any one of claims 39-42 wherein the unit of the material is positionable within a patient and the unit of material is degradable within the patient.

44. A system comprising:
a unit of a material that exhibits a color change that varies as an integral of an amount of ionizing radiation incident on the unit;
a casing about the unit; and
an illumination source within the casing, the illumination source positioned to illuminate the unit of the material;
an optical sensor within the casing positioned to detect one or more parameters indicative of a color of the unit of the material while the unit of the material is illuminated by the illumination source; and
a wireless communications interface configured to transmit information from the optical sensor to a remote receiver.

45. The system of claim 44 wherein the information includes unprocessed data from the optical sensor.

46. The system of any one of claims 44-45 further comprising a processor and a memory storing code to determine an amount of radiation exposure based upon the information from the optical sensor and to communicate the amount of radiation exposure through the wireless communications interface.

47. A system comprising:
a unit of a material that exhibits a color change that varies as an integral of an amount of ionizing radiation incident on the unit;
a casing disposed about the unit of the material; and
a connector coupled to the casing and releasably securable to a portable electronic device to optically couple the unit of the material to an illumination source powered by the portable electronic device and to an optical sensor powered by the portable electronic device.

48. The system of claim 47 wherein the connector includes an electrical coupling, the electrical coupling establishing electrical communication between a battery of the portable electronic device and one or more of the illumination source and the optical sensor when the connector is releasably secured to the portable electronic device.

49. The system of claim 48 the electrical coupling further establishing electrical communication between the battery of the portable electronic device and a processor when the connector is releasably secured to the portable electronic device, the processor coupled to one or more of the illumination source and the optical sensor.

50. The system of any one of claims 47-49 wherein the connector defines a recess releasably securable to the portable electronic device through a press fit.

51. The system of any one of claims 47-50 wherein the connector is releasably securable to the portable electronic device to optically couple a light emitted by the portable electronic device to the unit of the material.

52. The system of any one of claims 47-51 wherein the connector is releasably securable to the portable electronic device to optically couple a camera of the portable electronic device to the unit of the material.

53. The system of any one of claims 47-52 wherein the portable electronic device is one or more of a cellular phone, a handheld computer, an embedded system, and a mobile device.

54. A system comprising:
a unit of a material that exhibits a color change that varies as an integral of an amount of ionizing radiation incident on the unit;
a casing disposed about the unit of the material;
an illumination source and an optical detector within the casing;
circuitry within the casing configured to control the illumination source and capture an optical signal from the optical sensor; and
a connector coupled to the casing and releasably securable to an external device, the connector configured to provide power to the circuitry within the casing and to receive an electrical signal based on the optical signal from the optical sensor.

55. A system comprising:
a wearable garment;
a unit of a material securable in a fixed position relative to the wearable garment, the unit of the material exhibits a color change that varies as an integral of an amount of ionizing radiation incident on the unit; and
an optical coupling providing a terminal to optically couple an external device to the unit of the material.

56. The system of claim 55 wherein the unit of the material is releasably securable to the wearable garment.

57. The system of claim 56 wherein the unit of the material is releasably securable to the wearable garment through the optical coupling.

58. The system of any one of claims 55-57 wherein the wearable garment includes body armor.

59. The system of claim 55 wherein the wearable garment includes an elastic body suit including a plurality of units of the material at predetermined locations.

60. The system of claim 59 wherein the predetermined locations are selected to monitor radiation exposure during diagnostic imaging.

61. The system of claim 59 wherein the predetermined locations are selected to monitor radiation exposure during radiation therapy treatment.

62. The system of claim 55 wherein the wearable garment includes a glove suitable for use by a technician or a physician during radiation therapy on a patient.

63. A method comprising:
positioning a unit of a material at a location selected to measure incident radiation, wherein the material exhibits a color change that varies as an integral of an amount of ionizing radiation incident on the unit of the material;
illuminating the unit of the material with a light source;
detecting one or more parameters indicative of a color of the unit of the material; and
determining an exposure of the unit of the material to ionizing radiation based on the color of the unit of the material.

64. The method of claim 63 wherein illuminating the unit of the material includes exposing the unit of the material to a broadband light source.

65. The method of any one of claims 63 and 64 wherein illuminating the unit of the material includes exposing the unit of the material to a single wavelength light source.

66. The method of any one of claims 63-65 wherein the location is adjacent to tissue selected for radiation therapy.

67. The method of claim 66 further comprising exposing the tissue to a dose of ionizing radiation from a radiation source.

68. The method of claim 67 further comprising controlling at least one of a rate of the dose, an amount of the dose, and a direction of the dose based upon the exposure.
